(19) United States
(12) Patent Application Publication
(10) Pub. No.: US 2024/0165147 A1
(43) Pub. Date: May 23, 2024

(54) Methods for Treating Eyetear Film Deficiency

(71) Applicant: Bausch + Lomb Ireland Limited, Dublin (IE)

(72) Inventor: Annabelle Gallois-Bernos, Jacksonville, FL (US)

(21) Appl. No.: 17/991,083

(22) Filed: Nov. 21, 2022

(51) Int. Cl. A61K 31/728; A61K 9/00; A61K 9/107; A61K 47/44; A61P 27/04

(52) U.S. Cl. CPC A61K 31/728; A61K 9/0048; A61K 9/107; A61K 47/44; A61P 27/04

(57) ABSTRACT

The present invention relates to methods for treating tear film deficiencies in the eye by applying to the eye or the surrounding area an oil in water emulsion composition, optionally comprising a demulcent such as a hyaluronic acid compound.

### FIELD OF TECHNOLOGY

The present invention relates to methods for treating tear film deficiencies in the eye by applying to the eye or the surrounding area an oil in water emulsion composition, optionally comprising a demulcent such as a hyaluronic acid compound.

### BACKGROUND OF THE INVENTION

Dry eye is a debilitating condition causing dry-eye symptoms such as discomfort feelings on eyes and abnormalities in visual performance, associated with abnormalities in tear volume or quality, which may cause disorders on the surface of eyes such as cornea. The abnormalities in tear volume mainly refer to the condition of low lacrimal secretion, which is an index of dry eye based on the tear volume. Regarding the abnormalities in tear quality, abnormalities in tear ingredients such as few lipid or protein components contained in tears may deteriorate the stability of tear film, which may then cause dryness on the surface of eyes even with tear secretion.

When dry eye is associated with deficiencies or abnormalities in tear (or tear film) quality, dry eye may be evaporative dry eye, and may be associated with meibomiam gland dysfunction (MGD). MGD results from obstruction of meibomian glands and is a major cause of dry eye. Tear film stability is one of the ocular surface homeostatic signs and is key to preventing the desiccating stress that cause or perpetuate dry eye disease and that lead to compromise of ocular surface health.

Hyaluronic acid occurs naturally in the human body and has been shown to effectively treat symptoms of dry eye. Though long-term studies have yet to be done, treatment with sodium hyaluronate appears to accelerate recovery of the damaged cornea (Katsuyama I, Arakawa T. A convenient rabbit model of ocular epithelium damage induced by osmotic dehydration. J Ocul Pharmacol Tuer 2003 June; 19 (3): 281-9). Sodium hyaluronate eye drops increase precorneal tear film stability and corneal wettability, reduce the tear evaporation rate, and the healing time of corneal epithelium (Aragona P, Di Stefano G, Ferreri F, et al. Sodium hyaluronate eye drops of different osmolarity for the treatment of dry eye in Sjogren's syndrome patients. Br J Ophthalmol 2002 August; 86 (8): 879-84). Sodium hyaluronate can be found in AQuify contact lens comfort drops (CIBA Vision). However, it is difficult to incorporate water soluble polymers such as hyaluronic acid into ophthalmic oil-in-water emulsions.

### DESCRIPTION OF FIGURES

FIG. 1 shows results of MGD Rabbit Model for Determining Effect of Composition of Table 1 on NIBUT for Rabbit Groups 1 and 2 Measurements (average 3 measurements performed on healthy eye (control OS), Control OD (meibomian gland obstructed eye) and treated eye receiving Composition of Table 1 for Days 15-18 and first NIBUT of day on treated eye receiving Composition of Table 1 for Days 15-18).

### SUMMARY OF THE INVENTION

The present invention relates to methods of treating, preventing or reducing the symptoms associated with tear film deficiency in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition.

The present invention also relates to methods of increasing lacrimal secretion in a subject in one or both eyes of need thereof, comprising the step of administering a composition comprising an oil-in-water emulsion to the eye(s) and/or lacrimal gland(s) of the subject wherein the oil phase is present at a concentration of from about 0.01% w/v to about 10% w/v based on the total composition.

The present invention also relates to methods of increasing meibomian gland secretion in one or both eyes of a subject in need thereof, comprising the step of administering a composition comprising an oil-in-water emulsion to the eye(s) and/or the meibomian gland(s) of the subject wherein the oil phase is present at a concentration of from about 0.01% w/v to about 10% w/v based on the total composition.

The present invention also relates to methods of increasing tear film break up time in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition.

The present invention of relates to methods of treating, reducing, relieving or preventing low, decreased or decreasing tear film break up times in one or both eyes of a subject in need thereof due to meibomian gland dysfunction, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition.

The present invention further relates to methods of treating, reducing, relieving or preventing evaporative dry eye due to reduced or the inhibition or blockage of meibomian gland secretions in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition.

The present invention still further relates to methods of preventing, reducing and/or treating meibomian gland dysfunction in one or both eyes of a subject in need thereof, comprising the step of administering a composition comprising an oil-in-water emulsion to the eye(s) and/or the meibomian gland(s) of the subject wherein the oil phase is present at a concentration of from about 0.01% w/v to about 10% w/v based on the total composition.

The ophthalmic emulsions may, in accordance with the methods of the present invention, be dispensed to the eye of a subject up to four times or more per day per eye, as needed, to of reducing, relieving or preventing dry eye symptoms due to meibomian gland disfunction.

### DETAILED DESCRIPTION OF THE INVENTION

The methods and compositions of the present invention can comprise, consist of, or consist essentially of the essential elements and limitations of the invention described herein, as well any of the additional or optional features, components, or limitations described herein.

The term “comprising” (and its grammatical variations) as used herein is used in the inclusive sense of (and, interchangeably with the terms) “having” or “including” and not in the exclusive sense of “consisting only of.”

The terms “a” and “the” as used herein are understood to encompass the plural as well as the singular. Also, as used herein, “a,” “an,” “the,” “at least one,” and “one or more” are used interchangeably. Thus, for example, a composition that comprises “an” element can be interpreted to mean that the composition includes “one or more” such elements.

All percentages, parts and ratios are based upon the total weight of the composition of the present invention, unless otherwise specified.

All such weights as they pertain to the listed ingredients provided by way of example are based on the active level and, therefore, do not include carriers or by-products that may be included in commercially available materials, unless otherwise specified.

Unless otherwise indicated, all documents cited are, in relevant part, incorporated herein by reference; the citation of any document is not to be construed as an admission that it is prior art with response to the present invention. Furthermore, all documents incorporated herein by reference are only incorporated herein to the extent that they are not inconsistent with this specification.

In certain embodiments, the present invention as disclosed herein may be practiced in the absence of any compound, method step, or element (or group of compounds or elements) which is not specifically disclosed herein.

Unless defined otherwise, all technical and scientific terms used herein have the same meaning as commonly understood by one of ordinary skill in the art to which the invention belongs.

The methods of the invention are directed at preventing, reducing or relieving, or treating evaporative dry eye (symptoms thereof) due to meibomian gland disfunction (i.e., reduced or no secretions from one or more meibomian glands or poor quality secretions, resulting in tear film deficiency) comprising, consisting of, or consisting essentially of administering a safe and effective amount of an ophthalmic composition comprising, consisting of, or consisting essentially of ophthalmic oil-in-water emulsions. Also disclosed herein are methods of preventing, reducing or relieving, or treating tear film instability, enhancing lacrimal and/or meibomian gland secretion, increasing tear volume, or the prevention, reduction or treatment of meibomian gland dysfunction.

The term “emulsion” is used in its customary sense to mean a stable and homogenous mixture of two liquids which are immiscible or do not normally mix such as oil and water.

An “emulsifier” is a substance, such as a surfactant, which aids the formation of an emulsion. The terms “emulsifier” and “surfactant” are used interchangeably herein. In the context of the present invention, surfactant component means one or more surfactants that are present in the self-emulsifying composition and contribute to the self-emulsification.

The term “stable” is used in its customary sense and means the absence of creaming, flocculation, and phase separation.

The term “demulcent” is used in the usual sense and refers to an agent that relieves irritation of inflamed or abraded lens and/or eye surfaces.

The term “polar oil” means that the oil contains heteroatoms such as oxygen, nitrogen and sulfur in the hydrophobic part of the molecule.

The term “re-wetting” as used herein refers to the addition of liquid over at least a part, for example, at least a substantial part, of at least the anterior surface of a contact lens.

The term “tear film deficiency”, as used herein, means abnormalities in the tear film resulting in insufficient supply and/or excessive loss, and anomalous tear composition which reduce tear film stability or result in tear film instability. Such deficiency is typically caused by decreased (from typical amounts of) lacrimal and/or meibomian gland secretion and/or decreased (from typical amounts of) tear volume. Such decrease in lacrimal and/or meibomian gland secretion and/or decrease in tear volume can result in tear film breakup times of lower than 10 seconds—indicative of tear film instability. Tear film breakup times of 10 to 35 seconds are considered normal. (See Khurana, A K. “Diseases of lacrimal apparatus”. Comprehensive ophthalmology (6th ed.). Jaypee, The Health Sciences Publisher. p. 389. ISBN 978-93-5152-657-5.

The terms “treat” and “treatment” refer to the treatment of a patient afflicted with a pathological condition and refers to an effect that alleviates the condition by improving the stability or quality of the tear film, such as the thickness or balance of tear ingredients such as lipid or protein components and combinations of the foregoing. Treat and treatment also refer to an effect that results in the inhibition of the progress of the condition, and includes a reduction in the rate of progress, a halt in the rate of progress, amelioration of the condition. Treatment as a prophylactic measure (i.e., prophylaxis) is also included.

The meibomian glands are sebaceous glands along the edge of the eyelids that secret lipids that form the superficial layer of the tear film and protect the aqueous tear film layer from evaporating too quickly. The term “meibomian gland disfunction” means obstruction, blockage or loss of function of one or more meibomian glands. Meimbomian gland disfunction may be observed by at least one sign or symptom including corneal staining, ocular dryness, itching, burning, redness, conjunctivitis, light sensitivity, ocular discharge, thick meibum, a reduced quantity of meibum on the ocular surface. Ocular dryness may be characterized by measuring the tear film break up time as described herein. Meibomian gland dysfunction is a leading cause of evaporative dry eye and can worsen over time if left untreated. Evaporative dry eye is due to a deficient tear film lipid layer, which increases tear evaporation. It is caused by meibomian gland dysfunction, which occurs in over 85% of dry eye disease. (See Findlay Q, Reid K. Dry Eye Disease: When to Treat and When to Refer. Aust Prescr. 2018 October; 41(5):160-163.).

The term “ophthalmically compatible” means that the compound, component or solution does not have any substantial or significant detrimental effect on the (a) eye or ocular structure including the lids, ocular glands of the humans or animals to whom such compositions are administered, or (b) a contact lens being contacted or treated by the compositions of the methods of the present invention.

The term “lachrymal gland dysfunction” means partial or complete obstruction in the tear drainage system—typically caused by hormone changes, autoimmune disease, inflamed eyelid glands or allergic eye disease. The term “lachrymal secretion” means tears which cleans and refresh the eyes and include such components as electrolytes, water, proteins, and mucins.

The term “safe and effective amount” refers to an amount of an active ingredient that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is often measured by toxicity testing to determine the highest tolerable dose or the optimal dose of an active pharmaceutical ingredient needed to achieve the desired benefit. Studies that look at safety also seek to identify any potential adverse effects that may result from exposure to the drug. Efficacy is often measured by determining whether an active ingredient demonstrates a health benefit over a placebo or other intervention when tested in an appropriate situation, such as a controlled clinical trial or animal model testing.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous solution such as saline (e.g., 0.9% NaCl) or an aqueous pH buffered solution such as phosphate buffered saline (e.g., PBS).

The term “acceptable” with respect to a formulation, composition, or ingredient, as used herein, means that the beneficial effects of that formulation, composition, or ingredient on the general health of the patient being treated substantially outweigh its detrimental effects, to the extent any exist.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The term “ophthalmic device” refers to an object that resides in or on the eye. These devices can provide optical correction or may be cosmetic. Ophthalmic devices include (selected from or selected from the group consisting of), but are not limited to, soft contact lenses, intraocular lenses, overlay lenses, ocular inserts, punctual plugs, and optical inserts.

The compositions of the methods of the present invention comprise oil-in-water emulsions comprising an oil or oily component and an aqueous component which includes one or more emulsifiers or surfactants. The oil-in-water emulsions may comprise self-emulsifying surfactants.

### Oil or Oily Component:

The compositions of the methods of the present invention comprise one or more oils or oily substances/components which form an oil phase. The term “oils or oily”, as used herein means any non-polar liquid which is immiscible with aqueous liquids (i.e., not water-soluble or having a water solubility of less than 100 ug/ml). Any suitable oil or oily substance or combinations of oils or oily substances may be employed provided such oils and/or oily substances are effective in the compositions, and do not cause any substantial or significant detrimental effect to the human or animal to whom the composition is administered, or to the contact lens being treated, or the wearing of the treated contact lens, or to the wearer of the treated contact lens. The oily component may, for example, and without limitation, be polar in nature and naturally or synthetic derived. Natural oils may be obtained from plants or plant parts such as seeds or they may be obtained from an animal source such as Sperm Whale oil, Cod liver oil and the like. The oil may be a mono, di or triglyceride of fatty acids or mixtures of glycerides, such as Castor oil, Coconut oil, Cod-liver oil, Corn oil, Olive oil, Peanut oil, Safflower oil, Soybean oil and Sunflower oil. The oil may also be comprised of straight chain monoethylene acids and alcohols in the form of esters, such as Jojoba and Sperm Whale oil. The oil may be synthetic, such as silicone oil. The oil also may be comprised of water insoluble non-volatile liquid organic compounds, e.g., a racemic mixture of Vitamin E acetate isomers. Mixtures of the above oil types may also be used.

Oils which are natural, safe, have prior ophthalmic or other pharmaceutical use, have little color, do not easily discolor upon aging, easily form spread films and lubricate surfaces without tackiness are preferred. Castor oil and the like are preferred oils.

The oily component and the surfactant component(s) are advantageously chemically structurally compatible to facilitate self-emulsification of the emulsion. In the context of the present invention, surfactant component means one or two surfactants that are present in the self-emulsifying composition and contribute to the self-emulsification. The one or two surfactants must have an affinity for the selected oil or oils based upon non-covalent bonding interactions between the hydrophobic structures of the surfactant and the oil(s) such that self-emulsification can be achieved. In one aspect, affinity relates to the use of a polar oil with a surfactant of similar polarity. As the terms are used herein, a polar oil means that the oil contains heteroatoms such as oxygen, nitrogen and sulfur in the hydrophobic part of the molecule. In a preferred embodiment, the self-emulsifying emulsions described contain at least one polar oil.

When employed, the self-emulsifying, oil-in-water emulsions for the compositions used in the methods of the present invention are of two general types. The first type is a one surfactant system. The second type is a two surfactant system. In either case, what is required is that (1) the surfactant(s) must have an affinity for the selected oil or oils based upon non-covalent bonding interactions between the hydrophobic structures of the surfactant and the oil(s) such that self-emulsification can be achieved when requirement (2) is simultaneously met; and (2) the surfactant must have a chemical structure which is wedge or pie section-shaped, with the larger end of the wedge structure closer to the hydrophilic part of the surfactant structure. This wedge-shape is believed to induce spherical oil droplet curvature at the aqueous-oil interface due to the molecular self-assembly of adjacent surfactant wedges at the aqueous-oil interface. Thus, the geometry of the wedge-shaped surfactant molecules is intimately related to the oil droplet curvature. Steric repulsion in the aqueous phase between the hydrophilic parts of adjacent surfactant molecules facilitates this. Preferably, these hydrophilic parts consist of polyethyleneoxide chains of an appropriate length. Preferably, the polyethyleneoxide chains are from 7-20 ethyleneoxide units in length. When the aforementioned two structural requirements are met for a surfactant and oil(s) pair(s), an empirical test of self-emulsification is conducted while varying the concentrations of the surfactant and oil components. The empirical test of self-emulsification is conducted employing the methods of preparing self emulsifying emulsions described herein. An emulsion is considered to be acceptable when it appears to be homogeneous when observed by eye, without any appearance of flocculation, cream or phase separation between the aqueous and oil phase and also when the oil droplet size distribution of the emulsion meets particular product criteria for emulsion stability.

As a practical matter, a surfactant is a good candidate for the self-emulsifying oil-in-water emulsions described herein if the surfactant is able to form droplets of a size range of from 0.01 (or about 0.01) microns to 1 (or about 1) micron, or from 0.01 (or about 0.01) microns to 0.25 (or about 0.25) microns, or from 0.02 (or about 0.02) microns to 0.1 (or about 0.1) microns.

The oil(s)/oily component(s), including the oil/oily component of the compositions of the methods of the present invention can be present at concentrations of from about 0.01% w/v to about 10% w/v, or from about 0.05% w/v to about 5% w/v, or from about 0.1% w/v to about 1% w/v, or from about 0.2% w/v to about 0.5% w/v, based on the total composition.

### Aqueous Component

The compositions of the methods of the present invention also comprise an aqueous phase. The aqueous phase compositions of the methods of the present invention comprises water, mixtures of water and water-miscible solvents such as polyols.

As used herein, and unless otherwise indicated, the term “polyol” shall refer to any compound having at least two —OH groups. The polyols can be linear or circular, substituted or unsubstituted, or mixtures thereof, so long as the resultant complex is water-soluble and pharmaceutically acceptable. Such polyol compounds include sugars, sugar alcohols, sugar acids, uronic acids and mixtures thereof. In certain embodiments, the polyols are sugars, sugar alcohols and sugar acids, including, but not limited to: mannitol, glycerin (glycerol), propylene glycol, polyethylene glycol, sorbitol and mixtures thereof. In certain embodiments, the polyols are polysorbate 80, mannitol, sorbitol, propylene glycol, polyethylene glycol, glycerin or mixtures thereof. In certain embodiments, the polyol is glycerin, propylene glycol, polyethylene glycol or mixtures thereof. Preferably, the polyol is, polyethylene glycol. In other embodiments, the polyol is a combination of polyols such as glycerin and propylene glycol or glycerin and sorbitol. In certain embodiments, the polyol (or combinations thereof) can, optionally, be present in the composition of the methods of the present invention at concentrations of from about 0.01% w/v to about 2.0% w/v, optionally from about 0.1% w/v to about 1.7% w/v, optionally from about 0.2% w/v to about 1.5% w/v, or optionally from about 0.2% w/v to about 0.5% w/v, of the total composition.

In certain embodiments, the aqueous phase of the compositions of the methods of the present invention are isotonic, or slightly hypotonic in order to combat any hypertonicity of tears caused by evaporation and/or disease. This may require a tonicity adjusting agent. Tonicity adjusting agents are typically used in amounts ranging from about 0.001 to 2.5 w/v %. These amounts have been found to be useful in providing sufficient tonicity for maintaining ocular tissue integrity. Preferably, the tonicity agent(s) will be employed in an amount to provide a final osmotic value of 150 to 450 mOsm/kg, or between about 250 to about 330 mOsm/kg, or between about 270 to about 310 mOsm/kg. The compositions of the methods of the present invention (or aqueous component thereof) more preferably is substantially isotonic or hypotonic (for example, slightly hypotonic, e.g., about 240 mOsm/kg) and/or is ophthalmically acceptable. For example, the osmolality of the composition may be adjusted to approximate the osmotic pressure of normal tear fluid, which is equivalent to about 0.9 w/v % of sodium chloride in water. Examples of suitable tonicity adjusting agents include, without limitation, sodium, potassium, calcium and magnesium chloride; dextrose; glycerin; propylene glycol; mannitol; sorbitol and the like and mixtures thereof. Preferred tonicity-adjusting agents include, but are not limited to, mannitol, sodium chloride, glycerin, and mixtures thereof.

In certain embodiments, the aqueous phase of the compositions of the methods of the present invention may further include a buffer component which is present in an amount effective to maintain the pH of the composition (or aqueous component thereof) in the desired range.

In certain embodiments, the aqueous phase of the compositions of the methods of the present invention (or aqueous phase/component thereof) may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄. 7H₂O and NaH₂PO₄·H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v %, or varies from about 0.05 to about 0.5 w/v %, based on the total composition.

The type and amount of buffer are selected so that the formulation meets the functional performance criteria of the composition, such as surfactant and shelf-life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, or from about 6.5 to about 8.5, or from about 6.8 to about 8.0, or from about 7.3 to about 7.7 is also acceptable. In one embodiment, the aqueous phase of the compositions of the methods of the present invention have a pH of about 7.0.

The ophthalmic compositions of the methods of the present invention are generally be formulated as sterile or sterilized oil in water emulsions.

### Surfactants/Emulsifiers

The compositions of the methods of the present invention comprise surfactants/emulsions to form oil in water emulsions. In preferred embodiments, the one or two surfactants should be able to form a chemical structure which is wedge or pie section-shaped, with the larger end of the wedge structure having a larger effective radius, and closer to the hydrophilic parts of the surfactant structures than the smaller end of the wedge structure. That is, the part of the surfactant that is larger is oriented towards the aqueous phase and contains more atoms than the part of the surfactant that is oriented towards the oil phase. When the surfactant component includes two surfactants, the hydrophobic portion of the first surfactant may have a longer chain length than the hydrophobic portion of the second surfactant to promote formation of a wedge shape.

The surfactants useful to form the surfactant component in the present invention advantageously are water-soluble in the aqueous component. These surfactants are preferably non-ionic. Depending on the concentration of the oil phase, the amount of surfactant component present can vary over a wide range depending on a number of factors, for example, the other components in the composition and the like.. It is noted that additional surfactant(s) may be present in the self-emulsifying composition (in addition to the surfactant component) and still fall within the scope of the present invention if the additional surfactant(s) are present at a concentration such that they do not interfere with the self-emulsification.

The concentration of the total surfactant/emulsifier component is 0 or at least 0.01 w/w % (or about 0.01 w/w %), or from 0.05 w/w % (or about 0.05 w/w %), or from 0.1 w/w % (or about 0.1 w/w %), or from 0.2 w/w % (or about 0.2 w/w %), or from 0.25 w/w % (or about 0.25 w/w %) to no more than 5 w/w % (or about 5 w/w %), or 4 w/w % (or about 4 w/w %), or 3 w/w % (or about 3 w/w %), or 2 w/w % (or about 2 w/w %), or 1 w/w % (or about 1 w/w %) (or to less than 1 w/w % (or about 1 w/w %)), based on the total composition. In some embodiments of the methods of the present invention, the total amount of surfactant component is in the range of about 0.01 w/w % to about 10.0 w/w %, or of about 0.015 w/w % to about 7.0 w/w %, or of about 0.1 w/w % to about 5.0 w/w %, or about 0.15 w/w % to about 3.0 w/w %, or about 0.2 w/w % to about 1.0 w/w %, based on the total composition.

The ratio, for example, weight ratio, of the surfactant component to the oil/oily component in the present oil-in-water emulsions is selected to provide acceptable emulsion stability and performance, and preferably to provide a self-emulsifying oil-in-water emulsion. Of course, the ratio of surfactant component to oily component varies depending on the specific surfactants and oil or oils employed, on the specific stability and performance properties desired for the final oil-in-water emulsion, on the specific application or use of the final oil-in-water emulsion and the like factors.

Such surfactants are ophthalmically compatible, function as described herein and provide effective and useful ophthalmic compositions.

Examples of surfactants useful in the compositions of the methods of the present invention include polyethoxylated oils such as polyethylene glycol (PEG) castor oils. Polyethoxylated castor oil derivatives are formed by the ethoxylation of castor oil or hydrogenated castor oil with ethylene oxide. An example of a PEG castor oil is PEG40 hydrogenated castor oil (CRODURET 40, produced by Croda International Plc). A preferred example of a surfactant useful in forming a one surfactant system and oil pair is a PEG40 hydrogenated Castor oil and Castor oil.

The above-referenced PEG40 hydrogenated Castor oil is produced through the catalytic hydrogenation of Castor oil at the 9-carbon positions of the three ricinoleic acid glycerol ester chains, followed by ethoxylation of the three 12-hydroxy groups of the 12-hydroxystearic acid glycerol esters with about 13 ethoxy groups each.

Without being limited by theory, it is believed that self emulsification of Castor oil with the PEG40 hydrogenated castor oil occurs due to the folding of the 6-carbon alkyl chain distal to the ethoxylated 12-hydroxy group inwards against the remaining 10-carbon alkyl segment of the stearate ester group to form a wedge-shaped hydrophobic part of the molecule, the orientation of the ethoxy groups outwards into the water phase, the orientation of the wedge-shaped hydrophobic part of the molecule into the Castor oil phase (narrow part of the wedge facing inwards away from the aqueous phase) and the affinity of the wedge-shaped hydrophobic part of the molecule for Castor oil.

The optimal amount of the PEG hydrogenated castor oil to use in conjunction with Castor oil is about 0.8 w/w % PEG hydrogenated Castor oil for 1.0 w/w % Castor oil. Higher or lower amounts in conjunction with Castor oil can be used, however, depending upon the desired properties of the final emulsion. In general, the weight ratio of PEG hydrogenated castor oil to Castor oil is in the range of 0.6:1 w/w % to 0.8:1 w/w %.

PEG hydrogenated castor oil can be combined with additional surfactants such as Polysorbate-80 (Tween-80, polyoxyethylene (20) sorbitan mono-oleate) to create self-emulsifying emulsions comprised of two surfactants. In such compositions and without being limited by theory, self emulsification is believed to occur and be driven principally by the PEG hydrogenated castor oil. The second surfactant (e.g. polysorbate-80) does not interfere with the emulsifying action of the CRODURET 40 due to the similar chemical structures of the hydrophobic chains of Polysorbate-80 (oleic acid ester chains) and those of Castor oil (12-hydroxyoleic acid ester chains) and PEG hydrogenated castor oil (stearic acid ester chains). That is, the concentration of the non-interfering surfactant is low enough such that it does not interfere with the self-emulsification.

Two surfactants may also be selected to match particular oil or oils with respect to the ability of the surfactants to form a self-emulsifying oil-in-water emulsion for the dry eye treatments according to the invention. Both surfactants must each meet two chemical structural requirements to achieve self-emulsification: (1) each surfactant must have an affinity for the selected oil or oils based upon non-covalent bonding interactions between the hydrophobic structures of the surfactant and the oil(s) such that self emulsification can be achieved when requirement (2) is simultaneously met; and (2) the surfactant pair must be able to form a chemical structure which is wedge or pie section-shaped, with the larger end of the wedge structure closer to the hydrophilic parts of the surfactant structures. A preferred example of a surfactant pair which is compatible with the above described oils is the surfactant raw material Cremophor RH-40, from the BASF Corporation in Parsippany N.J., is comprised 75-78% of two surfactants: the trihydroxystearate ester of polyethoxylated glycerol and the hydroxystearate (bis)ester of mixed polyethylene glycols, along with 22-25% free polyethylene glycols. The Cremophor RH-40 raw material thus has two surfactants which are structurally related to each other and to Castor oil. It is believed that the combination of a surfactant with three ester chains with a surfactant with two ester chains, wherein all of the chains have an affinity for each other, allows the formation of a wedge-shaped structure in the presence of Castor oil wherein the two surfactants alternate at the oil droplet interface. Cremophor RH-60, also from BASF, is an example of another surfactant raw material comprised of two surfactants. Cremophor RH-60 is identical to Cremophor RH-40, with the exception that there is a higher derivatization with polyethyleneglycol with RH-60 than with RH-40.

Another example of a surfactant useful in the compositions of the methods of the present invention includes tocopherol polyethyleneglycol-succinate (TPGS, available from Eastman Chemical Company, Kingsport, Tenn.). TPGS can form a wedge with tocopherol in the narrow section, PEG in the outer section and succinate forming a covalent attachment between them.

More generic descriptions of the types of surfactants which can be used in the present invention include surfactants selected from: (a) at least one ether formed from 1 to 100 ethylene oxide units and at least one fatty alcohol chain having from 12 to 22 carbon atoms; (b) at least one ester formed from 1 to 100 ethylene oxide units and at least one fatty acid chain having from 12 to 22 carbon atoms; (c) at least one ether, ester or amide formed from 1 to 100 ethylene oxide units and at least one vitamin or vitamin derivative, and (d) mixtures of the above consisting of no more than two surfactants.

When employed, the self-emulsifying, oil-in-water emulsions for the compositions used in the methods of the present invention are of two general types. The first type is a one surfactant system. The second type is a two surfactant system. In either case, what is required is that (1) the surfactant(s) must have an affinity for the selected oil or oils based upon non-covalent bonding interactions between the hydrophobic structures of the surfactant and the oil(s) such that self emulsification can be achieved when requirement (2) is simultaneously met; and (2) the surfactant must have a chemical structure which is wedge or pie section-shaped, with the larger end of the wedge structure closer to the hydrophilic part of the surfactant structure. This wedge-shape is believed to induce spherical oil droplet curvature at the aqueous-oil interface due to the molecular self-assembly of adjacent surfactant wedges at the aqueous-oil interface. Thus, the geometry of the wedge-shaped surfactant molecules is believed to be intimately related to the oil droplet curvature. Steric repulsion in the aqueous phase between the hydrophilic parts of adjacent surfactant molecules facilitates this. Preferably, these hydrophilic parts consist of polyethyleneoxide chains of an appropriate length. The polyethyleneoxide chains are from 7-20 ethyleneoxide units in length. When the aforementioned two structural requirements are met for a surfactant and oil(s) pair(s), an empirical test of self emulsification is conducted while varying the concentrations of the surfactant and oil components. The empirical test of self emulsification is conducted employing the methods of preparing self emulsifying emulsions described herein. An emulsion is considered to be acceptable when it appears to be homogeneous when observed by eye, without any appearance of flocculation, cream or phase separation between the aqueous and oil phase and also when the oil droplet size distribution of the emulsion meets particular product criteria for emulsion stability.

As a practical matter, a surfactant is a good candidate for the self-emulsifying oil-in-water emulsions described herein if the surfactant is able to form droplets of a size range of 0.01 to 1 micron, or 0.05 to 0.25 microns.

Preferably, the emulsions of the present invention employ one or more, optionally two or more, surfactants selected from (or selected from the group consisting of) tocopherol polyethyleneglycol-succinate, PEG hydrogenated Castor oil, trihydroxystearate ester of polyethoxylated glycerol, hydroxystearate (bis)ester of mixed polyethylene glycols and mixtures thereof.

In certain embodiments, self-emulsifying oil-in-water emulsions can be formed with reduced amounts of dispersion mixing at shear speed, more preferably with substantially no dispersion mixing at shear speed. Dispersion mixing at shear speed is also known as mechanical homogenization. Mechanical homogenization to form an emulsion typically occurs at shear speeds greater than 1000 r.p.m., more typically at several thousand r.p.m., and even at 10,000 r.p.m. or more. In other words, the present self-emulsifying emulsions can be formed using reduced amounts of shear or using substantially no shear.

These emulsions employ molecular self-assembly methods to generate macromolecular oil droplet structures at the nanometer and sub-micron scale and thus represent an example of nanotechnology. The emulsions are easily prepared via molecular self-assembly in milliseconds to minutes. The emulsions can be filter sterilized and are storage-stable, for example, having a shelf life at about room temperature of at least about 2 years or more. In addition, the compositions of the methods of the present invention are advantageously easily sterilized, for example, using sterilizing techniques such as filtration sterilization, and eliminate, or at least substantially reduce, the opportunity or risk for microbial growth if the compositions become contaminated by inclusion of at least one anti-microbial agent.

Methods of Dosing and Treatment Regimes

In one aspect, described herein are methods of treating, reducing, relieving or preventing tear film instability in a mammal suffering from tear film instability in one or both eyes of the mammal by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing evaporative dry eye due to meibomian gland disfunction in a mammal suffering from evaporative dry eye in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing decreased or completely inhibited lacrimal gland secretions due to obstruction or blockage of lacrimal glands in a mammal suffering from decreased or completely inhibited lacrimal gland secretion in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing decreased or completely inhibited meibomian gland secretions due to obstruction or blockage of lacrimal glands in a mammal suffering from decreased or completely inhibited meibomian gland secretion in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing low, decreased or decreasing tear film breakup times (i.e., Tear film breakup times at (or trending) below 15 seconds, below 10 seconds or below 5 seconds) due to lacrimal gland dysfunction and/or meibomian gland dysfunction in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing meibomian gland dysfunction in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

In one aspect, described herein are methods of treating, reducing, relieving or preventing meibomian gland dysfunction in one or both eyes of a subject in need thereof by administering to at least one eye of said mammal a safe and effective amount of an ophthalmic solution comprising, consisting of, or consisting essentially of an oil-in-water emulsion wherein the oil phase is present at a concentration of from about 0.01% w/v to about 10% w/v of the total composition.

The compositions may be administered to the eye(s) of the mammal suffering from meibomian gland dysfunction, lacrimal gland dysfunction, or tear film deficiency as eye drops or indirectly through the use of contact lens soaked in the compositions of the present inventions. Contact lens care compositions include multipurpose cleaning, rinsing, disinfecting and storage compositions as well as rewetting, in-the-eye cleaning and other compositions for the contact lens. The compositions of the methods of the present invention may comprise additional components, for example other components for the treatment of dry eye (such as evaporative), or signs or symptoms of such dry eye, including dry eye symptoms due to meibomian gland disfunction, lacrimal gland dysfunction, or tear film deficiency.

The present invention further comprises methods of improving tear film stability in one or both eye(s) of an individual with meibomian gland disfunction comprising, consisting of, or consisting essentially of administering a safe and effective amount of an oil-in-water emulsion wherein the oil phase is present at a concentration of from about 0.01% w/v to about 10% w/v of the total composition.

The present inventor has found that by applying the certain compositions to the eyes and/or meibomian glands of mammals in accordance with the methods of present invention, the stability of tear films on the eyes can be significantly improved, as measured by non-invasive tear film breakup time. Specifically, the methods of the present invention can maintain (i.e., prevent further decrease in) the Non-Invasive Tear Break-Up Time (NITBUT) (a measure of tear film stability) so as to maintain the NITBUT within the range (e.g., within ±5% or ±3%) of NITBUT measurements obtained the day (or about the time—e.g., ±1 hour) of commencement of the methods of the present invention. Unless otherwise indicated in the present application, if multiple such NITBUT measurements are taken in a day, the first NITBUT measurement is used as the “measurement obtained the day (or about the time—e.g., ±1 hour) of commencement of the methods of the present invention” for purposes of the present application; this measurement, unless otherwise indicated herein, is considered the baseline for the NITBUT calculations in the present application—i.e., determining NITBUT increase in seconds or percentage increase

In certain embodiments, the methods of the present invention can increase the NITBUT beyond NITBUT measurement obtained the day (or about the time—e.g., ±1 hour) of commencement of the methods of the present invention by up to 1 (or about 1) seconds, up to 1.25 (or about 1.25) seconds or more, or by up to 1.5 (or about 1.5) seconds or more, or by up to 2 (or about 2) seconds or more, after administration, which increase, in certain embodiments, can be observed within (or after) about 10 days or more, 20 days or more, or 30 days or more, after such administration. In certain embodiments, the methods of the present invention can increase the NITBUT by at least 10% or more, at least 15% or more, at least 20% or more beyond NITBUT measurements obtained the day (or about the time—e.g., ±1 hour) of commencement of the methods of the present invention which increase, in certain embodiments, can be observed within (or after) about 10 days or more, 20 days or more, or 30 days or more after administration of the compositions disclosed herein according to the methods of the present invention.

Alternatively, the methods of the present invention can increase the NITBUT in a treated eye of a mammal (i.e., human or rabbit) beyond NITBUT measurement obtained in a nontreated eye of another mammal (i.e., human or rabbit) on the day(or about the time—e.g., ±1 hour) of commencement of the methods of the present invention by up to 1 (or about 1) seconds, up to 1.5 (or about 1.5) seconds or more, or by up to 2 (or about 2) seconds or more, or by up to 3 (or about 3) seconds or more, or by up to 4 (or about 4) seconds or more, after administration, which increase, in certain embodiments, can be observed within (or after) about 10 days or more, 20 days or more, or 30 days or more, after such administration. In certain of these such embodiments (i.e., certain of the embodiments of this paragraph), if multiple such NITBUT measurements are taken in a day, the average of such NITBUT measurements is used as the “measurement obtained the day (or about the time—e.g., ±1 hour) of commencement of the methods of the present invention” for purposes of this paragraph; such measurement being considered the baseline for the NITBUT calculations related to this paragraph—i.e., determining NITBUT increase in seconds or percentage increase. In these embodiments, the methods of the present invention can increase the NITBUT in a treated eye of a mammal (i.e., human or rabbit) by, at least 10% or more, at least 20% or more, at least 30% or more, at least 40% or more, at least 50% or more, at least 60% or more, at least 70% or more, at least 80% or more, at least 90% or more, or at least 100% or more, beyond NITBUT measurements obtained in a nontreated eye of another mammal (i.e., human or rabbit) on the day (or about the time—e.g., ±1 hour) of commencement of the methods of the present invention which increase, in certain embodiments, can be observed within (or after) about 10 days or more, 20 days or more, or 30 days or more after administration of the compositions disclosed herein according to the methods of the present invention.

The ophthalmic compositions may be dispensed to the eye as needed for reducing, relieving or preventing evaporative dry eye symptoms due to meibomian gland disfunction, lacrimal gland dysfunction and/or tear film instability.

When the ophthalmic compositions of the present invention are administered to the eye as eye drops, they are administered at least once per day, at least twice or more, at least three times or more, at least four times or more, at least five or more times, or at least six times or more per day, as needed, to the eye or eyes experiencing tear film instability, meibomian gland dysfunction, or lacrimal dysfunction or signs or symptoms thereof. The intervals between such times of administration can be every 12 hours, every 8 hours, every 6 hours and every 4 hours, as needed. In certain embodiments, reduction in certain of the signs/symptoms of such meibomian gland disfunction, lacrimal gland dysfunction and/or tear film instability may be observed within 1 day, within 2 days, within 3 days, within 4 days, within 5 days, within 6 day, within 7 days, within 8 days, within 9 days, within 10 days, within 15 day, within 20 days, within 25 days, or within 30 days of starting administration of the compositions according to the methods of the present invention.

The integration of oil-in-water emulsions into eye drops for treating tear film instability, enhancing lacrimal and/or meibomian gland secretion, increasing tear volume, or the prevention, reduction or treatment of meibomian gland dysfunction may also add the additional utility of prevention treatment or reduction of tear film deficiencies and contact lens water loss by providing an oil layer at the air-tear interface or additionally at the contact lens-tear interface when a contact lens is present.

The present invention provides for methods of using the emulsion compositions as described elsewhere herein. In one embodiment, the present methods comprise administering a composition of the invention to one or both eye(s) of a subject, for example, a mammal or a human, in an amount and under conditions effective to provide a benefit to the eye such as tear film stabilization, enhanced lacrimal and/or meibomian gland secretion, increased tear volume or the prevention, reduction or treatment of meibomian gland dysfunction or the signs or symptoms of meibomian gland dysfunction, lacrimal gland dysfunction, or tear film deficiency.

In an embodiment, the present methods comprise contacting a contact lens with a composition of the present invention in an amount and at conditions effective to provide at least one of the above-mentioned benefits to the wearer of the contact lens. In this embodiment, the composition of the methods of the present invention is employed as at least a portion of a contact lens care composition.

The components used in accordance with the present invention are selected to be effective in the emulsion compositions of the methods of the present invention and to have no substantial or significant deleterious effect, for example, on the compositions, on the use of the compositions, on the contact lens being treated, on the wearer of the treated lens, or on the human or animal in whose eye the composition is placed.

### Optional Components

The compositions used in the methods of the present invention may optionally further include compounds which act as either demulcents and/or viscosity modifying agents/components. Preferably the demulcents and/or viscosity modifying agents/components are soluble in the aqueous phase.

Compounds preferred for use as viscosity modifying agents include hyaluronic acid compounds (HA), cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers (e.g. Carbopol®); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the compositions.

Compounds preferred for use as demulcents include hyaluronic acid (or hyaluronate salts), carboxymethylcellulose, other cellulose polymers (e.g., carboxy methyl cellulose methylcellulose, hydroxyethylcellulose, hydroxypropylcellulose, hydroxypropyl methylcellulose (HPMC) and methyl ethyl cellulose), dextran 70, gelatin, polyethylene glycols (e.g., PEG 300 and PEG 400), polyvinyl alcohol, polyvinylpyrrolidone (PVP) and the like and mixtures thereof, may also be incorporated into the compositions used in the methods of the present invention.

In more preferred embodiments, demulcents may further include water soluble polymers such as hyaluronic acid (or hyaluronate salts), polyvinylpyrrolidone (PVP), and hydroxypropyl methylcellulose (HPMC), polyethylene glycols (e.g., PEG 300 and PEG 400) and salts thereof and mixtures thereof.

The concentration of such demulcents or viscosity modifiers will typically vary between about 0.01% w/v to about 5% w/v of the total composition, although higher (or lower) concentrations of certain of the viscosity modifiers may be employed based on the desired viscosity for the composition.

Particularly preferred compounds showing demulcent activity are hyaluronic acid compounds (HA). Such compounds are natural polymers. As used herein, the term “hyaluronic acid compound (HA)” means hyaluronic acid and any of its hyaluronate salts, including, for example, sodium hyaluronate (the sodium salt), potassium hyaluronate, magnesium hyaluronate, and calcium hyaluronate.

HA is a polymer consisting of simple, repeating disaccharide units (glucuronic acid and N-acetyl glycosamine). It is made by connective tissue cells of all animals and is present in large amounts in such tissues as the vitreous humor of the eye, the synovial fluids of joints, and the roostercomb of chickens. One method of isolating HA is to process tissue such as roostercombs. HA isolated from natural sources can be obtained from commercial suppliers, such as Biomatrix, Anika Therapeutics, ICN, and Pharmacia.

Another method of producing HA is via fermentation of bacteria, such as streptococci. The bacteria are incubated in a sugar rich broth and excrete HA into the broth. HA is then isolated from the broth and impurities are removed. The molecular weight of HA produced via fermentation may be altered by the sugars placed in the fermentation broth. HA produced via fermentation can be obtained from companies such as Bayer, Genzyme, and Fidia.

In its natural form, HA has a molecular weight in the range of 5×10⁴ up to 1×10⁷ daltons. Its molecular weight may be reduced via a number of cutting processes such as exposure to acid, heat (e.g. autoclave, microwave, dry heat), or ultrasonic waves. HA is soluble in water and can form highly viscous aqueous solutions. In the compositions of the present invention, the molecular weight for the HA is 8×10⁴ up to 2×10⁶ daltons, or 1.44×10⁶ to 1.66×10 6 daltons.

The HA can be present in the compositions of present method at concentrations either alone or in combination with a second demulcent at concentrations of at a concentration of at least 0.001% (or about 0.001) w/w of the total composition.

Typically, HA is present at a concentration of from 0.01% (or about 0.01%) w/w to 1% w/w, or from 0.05% (or about 0.05%) w/w to 0.5% (or about 0.5%) w/w, or from 0.1% (or about 0.1%) w/w to 0.3% (or about 0.3%) w/w, based on weight of the total composition. In certain embodiments, HA can be present as higher concentrations. HA is present at 0.15% (or about 0.15%) w/w to 0.25% (or about 0.01%) w/w, based on the weight of the total composition.

In certain embodiments, the compositions of the methods of the present invention further include sequestering agents or components in order to, and in an amount effective to, bind metal ions, which, for example, might otherwise stabilize cell membranes of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v %. Examples of useful sequestering components include, without limitation ethylene-diaminetetraacetic acid (EDTA) and its potassium or sodium salts and low molecular weight organic acids such as citric and tartaric acids and their salts, e.g., sodium salts.

In addition to demulcent/viscosity modifying, tonicity and buffer components mentioned above, the present compositions may include one or more other materials, for example, as described elsewhere herein, in amounts effective for the desired purpose, for example, to treat contact lenses and/or ocular tissues, for example, to provide a beneficial property or properties to contact lenses and/or ocular tissues, contacted with such compositions.

The compositions used in the methods of the present invention are useful, for example, as a carrier or vehicle, for the delivery of at least one additional therapeutic agent to or through the eye. Any suitable therapeutic component may be included in the present compositions provided that such therapeutic component is compatible with the remainder of the composition, does not unduly interfere with the functioning and properties of the remainder of the composition, is effective, for example, to provide a desired therapeutic effect, when delivered in the present composition and is effective when administered to or through the eye.

Optionally, an effective amount of a desired second therapeutic agent or component preferably is physically combined or mixed with the other components of a composition of the present invention to form a therapeutic component-containing composition within the scope of the present invention.

The type of second therapeutic agent or agents used will depend primarily on the therapeutic effect desired, for example, the disease or disorder or condition to be treated. These therapeutic agents or components include a broad array of drugs or substances currently, or prospectively, delivered to or through the eye in topical fashion or otherwise. Examples of useful additional therapeutic components which may be used in conjunction with a treatment for dry eye include, but not limited to:
(1) anti-infective and anti-microbial substances including quinolones, such as ofloxacin, ciprofloxacin, norfloxacin, gatifloxacin and the like; beta-lactam antibiotics, such as cefoxitin, n-formamidoyl-thienamycin, other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin sulfonamides and the like; aminoglycoside antibiotics, such as gentamycin, kanamycin, amikacin, sisomicin, tobramycin and the like; naladixic acid and analogs thereof and the like; antimicrobial combinations, such as fluealanine/pentizidone and the like; nitrofurazones; and the like and mixtures thereof;
(2) anti-allergy agents, antihistaminics, anti-hypertensive agents and decongestants, such as pyrilamine, chlorpheniramine, phenylephrine hydrochloride, tetrahydrazoline hydrochloride, naphazoline hydrochloride, oxymetazoline, antazoline, and the like and mixtures thereof;
(3) anti-inflammatories, such as cortisone, hydrocortisone, hydrocortisone acetate, betamethansone, dexamethasone, dexamethasone sodium phosphate, prednisone, methylprednisolone, medrysone, fluorometholone, fluocortolone, prednisolone, prednisolone sodium phosphate, triamcinolone, sulindac, salts and corresponding sulfides thereof, and the like and mixtures thereof;
(4) non-steroid anti-inflammatory drug (NSAID) components, such as those which do or do not include a carboxylic (—COOH) group or moiety, or a carboxylic derived group or moiety; NSAID components which inhibit, either selectively or non-selectively, the cyclo-oxygenase enzyme, which has two (2) isoforms, referred to as COX-1 and COX-2; phenylalkoanoic acids, such as diclofenac, flurbiprofen, ketorolac, piroximcam, suprofen and the like; indoles such as indomethacin and the like; diarylpyrazoles, such as celecoxib and the like; pyrrolo pyrroles; and other agents that inhibit prostaglandin synthesis and the like and mixtures thereof;
(5) miotics and anticholinergics, such as echothiophate, pilocarpine, physostigmine salicylate, diisopropylfluorophosphate, epinephrine, dipivolyl epinephrine, neostigmine, echothiopate iodide, demecarium bromide, carbachol, methacholine, bethanechol, and the like and mixtures thereof;
(6) mydriatics, such as atropine, homatropine, scopolamine, hydroxyamphetamine, ephedrine, cocaine, tropicamide, phenylephrine, cyclopentolate, oxyphenonium, eucatropine, and the like and mixtures thereof;
(7) antiglaucoma drugs, for example, prostaglandins, such as those described in U.S. Pat. Nos. 6,395,787 and 6,294,563, which are herein incorporated by reference in their entirety, adrenergic agonists such as quinoxalines and quinoxaline derivatives, such as (2-imidozolin-2-ylamino)quinoxaline, 5-halide-6-(2-imidozolin-2-ylamino)quinoxaline, for example, 5-bromo-6-(2-imidozolin-2-ylamino)quinoxaline and brimonidine and its derivatives, such as those described in U.S. Pat. No. 6,294,563, which is herein incorporated by reference in its entirety and the like, timolol, especially as the maleate salt and R-timolol and timolol derivatives and a combination of timolol or R-timolol with pilocarpine and the like; epinephrine and epinephrine complex or prodrugs such as the bitartrate, borate, hydrochloride and dipivefrin derivatives and the like; hyperosmotic and dipivefrin derivatives and the like; betaxolol, hyperosmotic agents, such as glycerol, mannitol and urea and the like and mixtures thereof;
(8) antiparasitic compounds and/or anti-protozoal compounds, such as ivermectin; pyrimethamine, trisulfapyrimidine, clindamycin and corticosteroid preparations and the like and mixtures thereof;
(9) antiviral compounds, such as acyclovir, 5-iodo-2′-deoxyuridine (IDU), adenosine arabinoside (Ara-A), trifluorothymidine, interferon and interferon inducing agents, such as Poly I:C and the like and mixtures thereof;
(10) carbonic anhydrase inhibitors, such as acetazolamide, dichlorphenamide, 2-(p-hydroxyphenyl)thio-5-thiophenesulfonamide, 6-hydroxy-2-benzothiazole-sulfonamide 6-pivaloyloxy-2-benzothiazolesulfonamide and the like and mixtures thereof;
(11) anti-fungal agents, such as amphotericin B, nystatin, flucytosine, natamycin, and miconazole and the like and mixtures thereof;
(12) pain-relieving and anesthetic agents, such as etidocaine, cocaine, benoxinate, dibucaine dydrochloride, dyclonine hydrocholoride, naepaine, phenacaine hydrochloride, piperocaine, proparacaine hydrochloride, tetracaine hydrochloide, hexylcaine, bupivacaine, lidocaine, mepivacaine and prilocaine and the like and mixtures thereof;
(13) ophthalmic diagnostic agents, such as
(a) those used to examine the retina, such as choride-sodium fluorescein and the like and mixtures thereof;
(b) those used to examine the conjunctiva, cornea and lacrimal structures, such as fluorescein and rose Bengal and the like and mixtures thereof; and
(c) those used to examine abnormal pupillary responses such as methacholine, cocaine, adrenaline, atropine, hydroxyamphetamine and pilocarpine and the like and mixtures thereof;
(14) ophthalmic agents used as adjuncts in surgery, such as alpha-chymotrypsin, and hyaluronidase and the like;
(15) chelating agents, such as ethylenediamine tetraacetate (EDTA) and deferoxamine and the like; and mixtures thereof;
(16) immunosuppressive agents and anti-metabolites, such as methotrexate, cyclophosphamide, 6-mercaptopurine, cyclosporins such A through I and azathioprine and the like; and mixtures thereof;
(17) angiostatic agents;
(18) muco-secretogogue agents;
(19) proteins and growth factors such as epidermal growth factor;
(20) vitamins and vitamin derivatives such as vitamins A, B12, C, D, E, folic acid and their derivatives;
(21) combinations of the above such as antibiotic/anti-inflammatory as in neomycin sulfate-dexamethasone sodium phosphate, quinolone-NSAID and the like; and concomitant anti-glaucoma therapy, such as timolol maleate-aceclidine and the like.

When a second therapeutic component is present in the compositions used in the methods of the present invention, the amount of such therapeutic component in the composition preferably is effective to provide the desired therapeutic effect to the human or animal to whom the composition is administered, without diminishing the treatment effect of the methods of the present invention.

Typically, when a second therapeutic component is present, the compositions comprising oil-in-water emulsions of the present invention may contain from or at least about 0.001%, for example, about 0. 01%, to about 5% (w/v) of the therapeutic component, e.g., medicament or pharmaceutical, on a weight to weight basis. Thus, for example, from one drop of liquid composition which contains about 25 mg of composition, one would obtain about 0.0025 mg to about 1.25 mg of therapeutic component.

The particular therapeutic component, e.g., drug or medicament, used in the pharmaceutical compositions of this invention is the type which a patient would require or benefit from for the treatment, e.g., pharmacological treatment, of a condition which the patient has or is to be protected from or from which the patient is suffering. For example, if the patient is suffering from glaucoma, the drug of choice may be timolol and/or one or more other anti-glaucoma components.

It is within the knowledge of one skilled in the art to determine the correct amounts of therapeutic component, e.g., drug, to be added to a composition of the invention in order to assure the efficacious delivery of the desired therapeutic component.

All types of contact lenses may be used in association with the methods of the present invention. For example, such contact lenses may be soft, rigid and soft or flexible gas permeable, silicone hydrogel, silicon non-hydrogel and conventional hard contact lenses.

The present compositions used in the methods of the present invention may further comprise antimicrobial agents (i.e., preservatives or disinfectants) to preserve the compositions from microbial contamination and/or disinfect contact lenses. The amount of the disinfectant component present in the liquid aqueous medium is effective to disinfect a contact lens placed in contact with the composition.

For example, the disinfectant component may include, but is not limited to, quaternary ammonium salts used in ophthalmic applications such as poly[dimethylimino-w-butene-1,4-diyl]chloride, alpha[4-tris(2-hydroxyethyl)ammonium]-dichloride (chemical registry number 75345-27-6, available under the trademark Polyquaternium 1® (from Onyx Corporation), poly(oxyethyl(dimethyliminio)ethylene dmethyliminio) ethylene dichloride sold under the trademark WSCP by Buckman laboratories, Inc. in Memphis, Tenn., benzalkonium halides, salts of alexidine, alexidine-free base, salts of chlorhexidine, hexetidine, alkylamines, alkyl di- and tri-amine, tromethamine(2-amino-2-hydroxymethyl-1, 3propanediol), hexamethylene biguanides and their polymers, cetylpyridinium chloride, cetylpyridinium salts, antimicrobial polypeptides, and the like and mixtures thereof. A particularly useful disinfectant component is selected from one or more (mixtures) of polyhexamethylene biguanide (PHMB), Polyquaternium-1, ophthalmically acceptable salts thereof, and the like and mixtures thereof.

The salts of alexidine and chlorhexidine can be either organic or inorganic and are typically disinfecting gluconates, nitrates, acetates, phosphates, sulphates, halides and the like. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which is incorporated in its entirety by reference herein.

The disinfectant components useful in the present invention are preferably present in the present compositions in concentrations in the range of about 0.00001% to about 2% (w/v).

More preferably, the disinfectant component is present in the present compositions at an ophthalmically acceptable or safe concentration such that the user can remove the disinfected lens from the composition and thereafter directly place the lens in the eye for safe and comfortable wear.

When a contact lens is desired to be disinfected by a disinfectant component, an amount of disinfectant effective to disinfect the lens is used. An effective amount of the disinfectant reduces the microbial burden on the contact lens by one log order, in three hours. In preferred embodiment, an effective amount of the disinfectant reduces the microbial load by one log order in one hour.

The disinfectant component is preferably soluble in the compositions of the methods of the present invention (or aqueous component thereof).

The present compositions may include an effective amount of a preservative component. Any suitable preservative or combination of preservatives may be employed. Examples of suitable preservatives include, without limitation, chlorous acid compounds such as sodium chlorite or stabilized chlorine dioxide (SCD) Purogene® material (PUROGENE is a trademark of BioCide International, Inc. Norman, Okla., U.S.A., and is also available as Purite® which is a trademark of Allergan, Inc.), polyhexamethylene biguanide (PHMB), Polyquaternium-1, ophthalmically acceptable salts thereof, and the like and mixtures thereof, benzalkonium chloride, methyl and ethyl parabens, hexetidine and the like and mixtures thereof. The amount of preservative components included in the present compositions are such to be effective in preserving the compositions and can vary based on the specific preservative component employed, the specific composition involved, the specific application involved, and the like factors. Preservative concentrations often are in the range of about 0.00001% to about 0.05% or about 0.1% (w/v) of the composition, although other concentrations of certain preservatives may be employed.

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

Preferred for use herein is a chlorous acid compound including (selected from or selected from the group consisting of), but not limited to, potassium chlorite, sodium chlorite, calcium chlorite, magnesium chlorite and mixtures thereof. In a preferred embodiment, the chlorite compound comprises sodium chlorite.

The present compositions may comprise effective amounts of one or more additional components. For example, one or more conditioning components or one or more contact lens wetting agents and the like and mixtures thereof may be included. Acceptable or effective concentrations for these and other additional components in the compositions of the invention are readily apparent to the skilled practitioner.

When any component is included, it is preferably compatible under typical use and storage conditions with the other components of the composition. The preparation of the oil-in-water emulsion compositions used in the methods of the present invention are generally as follows:

Non-emulsifying agents which are water soluble components, including the water-soluble demulcent(s), are dissolved in the aqueous (water) phase and oil-soluble components including the emulsifying agents are dissolved in the oil phase. The two phases (oil and water) are separately heated to an appropriate temperature. This temperature is the same in both cases, generally a few degrees to 5 to 10 degrees above the melting point of the highest melting ingredients in the case of a solid or semi-solid oil or emulsifying agent in the oil phase. Where the oil phase is liquid at room temperature, a suitable temperature is determined by routine experimentation with the melting point of the highest melting ingredients in the aqueous phase. In cases where all components of either the oil or water phase are soluble in their respective phase at room temperature, no heating may be necessary. The temperature must be high enough that all components are in the liquid state but not so high as to jeopardize the stability of the components. A working temperature range is generally from about 20° C. to about 70° C. To create an oil-in-water emulsion, the final oil phase is gently mixed into either an intermediate, preferably de-ionized water phase, or the final aqueous phase to create a suitable dispersion and the product is allowed to cool with or without stirring. In the case wherein the final oil phase is first gently mixed into an intermediate water phase, this emulsion concentrate is thereafter mixed in the appropriate ratio with the final aqueous phase. The final aqueous phase includes the water-soluble polymer as well as other aqueous-soluble components. In such cases, the emulsion concentrate and the final aqueous phase need not be at the same temperature or heated above room temperature, as the emulsion has already been formed at this point. In a preferred embodiment, the particles are between 0.08 and 0.18 microns in size before passing to the next step. A Horiba LA-920 particle size analyzer may be used according to the manufacturer's instructions for this purpose.

In the next step, the formed particles may be mixed with other aqueous components such as water, one or more demulcents and buffer (preferably boric acid based). Optionally, electrolytes, such as calcium chloride dihydrate, magnesium chloride hexahydrate, potassium chloride and sodium chloride, and Kollidon 17 NF may be added. While the electrolytes are not necessary to form the emulsions, they are very helpful to preserve ocular tissue integrity by maintaining the electrolyte balance in the eye. Likewise, the buffer is not critical, but, if included, a boric acid/sodium borate system is preferred.

The oil-in-water emulsion compositions of the methods of the present invention can be sterilized after preparation using autoclave steam sterilization, UV sterilization and gamma electron beam sterilization, or can be sterile filtered or sterilized by any other means known in the art. Sterilization employing a sterilization filter can be used when the emulsion droplet (or globule or particle) size and characteristics allows. The droplet size distribution of the emulsion need not be entirely below the particle size cutoff of the sterile filtration membrane to be filterable by the sterile-filter. In cases where the droplet size distribution of the emulsion is above the particle size cutoff of the sterile filtration membrane, the emulsion needs to be able to deform or acceptably change while passing through the filtrating membrane and then reform after passing through. This property is easily determined by routine testing of emulsion droplet size distributions and percent of total oil in the compositions before and after filtration. Alternatively, a loss of a small amount of larger droplet-sized material may be acceptable.

The emulsion compositions of the methods of the present invention are filter sterilized one or more times using a 0.22 micron filter. In a preferred embodiment, 98-99% of the emulsion should pass through the 0.22 micron filter. Note that particles larger than 0.22 micron may pass through by altering their shape temporarily. In a preferred embodiment, the material is then tested to verify the effectiveness of the sterilization step. Storage is below 25° C. in order to maintain stability. Thereafter, the emulsions are aseptically filled into appropriate containers.

The compositions used in the methods of the present invention can stored and/or dispensed from multidose containers or from single dose containers. The multidose or single dose containers, in each case, can contain preservatives as described herein or can be free of preservatives.

It will be understood by those of skill in the art that numerous and various modifications can be made without departing from the spirit of the present invention. Therefore, it should be clearly understood that the following examples are illustrative only and are not intended to limit the scope of the present invention.

### EXAMPLES

The compositions used in the methods of the present invention described in following examples illustrate specific embodiments of compositions used in the present invention, but are not intended to be limiting thereof. Other modifications can be undertaken by the skilled artisan without departing from the spirit and scope of this invention.

### Example 1

Table 1 shows a hyaluronic acid emulsion (with lipids/oil) composition of the present invention suitable for direct application to the eyes, which composition can be prepared using conventional mixing technology.

TABLE 1

| Ingredient | (% w/v) |
|---|---|
| Sodium Hyaluronate | 0.200 |
| Sodium Chloride | 0.050 |
| Potassium Chloride | 0.140 |
| Boric Acid | 0.600 |
| Sodium Borate Decahydrate | 0.070 |
| Polyethylene Glycol 400 | 0.250 |
| Castor Oil (Super-refined) | 0.313 |
| PEG40 hydrogenated castor oil | 0.250 |
| (Croduret) | |
| Sodium Chlorite | 0.014 |
| Purified Water | QS |

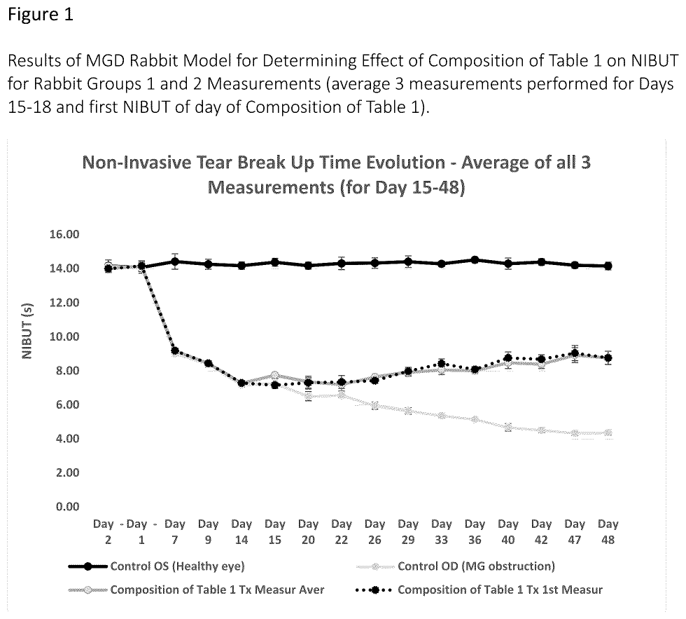

The meibomian gland dysfunction (MGD) rabbit model (Gallois-Bernos et al, ARVO poster 2021: Development of a meibomian gland dysfunction model in the rabbit—https://iovs.arvojournals.org/article.aspx?articleid=2774732) testing was performed evaluating the composition of Table 1 as follows:
10 New Zealand Rabbits (males, at least 2.5 kg)
   5 day acclimation period
   Group 1: 5 males, Control, Full cautery of meibomian gland in right eye; left eye was not cauterized. No eye drops were administered to group 1 rabbits
   Group 2: 5 males, Test, Full Cautery of Meibomian gland in both left eye and right eye. Eye drops starting Days 15-47, in OD (composition of Table 1) three times per day
Pre-cauterization steps:
   the test rabbits were sedated with appropriate anesthesia using 35 mg/kg ketamine and 5 mg/kg xylazine (intramuscular) and/or 1-3% Isoflurane (inhalation) prior to the cauterization on Day 0.
   The corneas were topically anaesthetized with 0.5% proparacaine HCl ophthalmic solution.
Cauterization of meibomian gland orifices was performed under a surgical microscope with a standard ophthalmic cautery with care taken to avoid thermal damage and touching the surrounding tissues. Orifice closure was confirmed (absence of visible meibomian gland orifice/absence of oil on the lid margin and in the tear film, no oil expressed from the glands with digital pressure). The day of cauterization was designated as Day 0. The only the left eye of each rabbit in Group 1 was cauterized and both eyes of each rabbit in Group 2 were cauterized. The rabbits were treated with buprenorphine hydrochloride 0.03 mg/kg twice by subcutaneous injections on the day of cauterization. (Day 0). (Balanced salt solution (BSS) was applied to both eyes to avoid drying of the eyes under anesthesia.)
Post-cauterization steps:
   After the cauterization of meibomian gland orifices, balanced salt solution (BSS) was administered to the surface of the rabbit eyes to keep them moist.
   Animals were treated with buprenorphine hydrochloride 0.03 mg/kg twice by
   subcutaneous injections on the day of cauterization (Day 0).
   The right eye per animal in Group 2 had instilled therein the eye drop of Table 1.
   The composition of Table 1 was instilled in the right eye (OD) three times per day from Day 15 to Day 47.
Test eye application solutions:
   Both eyes per rabbit in Group 1 were left untreated as controls.
   The eyes of the rabbits in Group 2 were treated as follows starting on Day 15 to Day 47:
      30 μL of composition of Table 1 was instilled in the right eye (OD) three times per day (with at least 3 hour intervals between each dose)
Clinical observations were conducted pre-study, daily (post treatment-dose) and pre-sacrifice.
Body weights were conducted pre-study and pre-sacrifice.
Draize scores were conducted starting pre-study (Day −2), daily (post treatment-dose) and pre-sacrifice.
Ocular examinations (slit lamp only) were performed Days −1, 7, 14, 15, 19, 22, 29, 36, 43 and 47
(pre-sacrifice). (The performed days had deviation ±2 days if the days are falling into the weekend.)
Fluorescein Staining was performed on Days −1, 14, 22, 29, 36, 43 and 47 (pre-sacrifice). (The performed days had deviation ±2 days if the days are falling into the weekend.)
Non-Invasive Tear Film Breakup Time (NIBUT) in all animals was performed on Days −2, −1, 7, 9, 14 (i.e., on days prior to starting test solution dosing regimen) and on Days 15, 19, 22, 26, 29, 33, 36, 40, 43 and 47 (i.e., on days after commencement of test solution dosing regimen). (The performed days had deviation ±2 days if the days are falling into the weekend.)
   Prior to test dosing regimen, both eyes in animals had NIBUT measurement performed once on Days −2, −1, 7, 9, and 14 prior to test solution dosing administration using a slit lamp (SL-17 Handheld Portable Slit Lamp from Optics Incorporated) with a cobalt blue filter in conjunction with a Tearscope Plus from Keeler. Each eye was blinked three times before the measurement. The eye was placed in proper position for measurement while closed. The Tearscope Plus and the slit lamp were used at the same time to focus on the tear film of the eye and then the time from upstroke of the last blink to the first tear film break (or dry spot formation) was recorded as NIBUT (in seconds). The measurements were performed ±2 days if the scheduled day fell on a weekend or holiday.
   After commencement of the test dosing regimen, both eyes in animals had NIBUT measurement performed three times on Days 15, 19, 22, 26, 29, 33, 36, 40 and 43, and once at termination on day 47 using the slit lamp (SL-17 Handheld Portable Slit Lamp from Optics Incorporated) with a cobalt blue filter in conjunction with a Tearscope Plus from Keeler. NIBUT was performed three times per performed day on each eye: before first test solution instillation, after second test solution instillation, and before third test solution instillation. Each eye was blinked three times before the measurement. The eye was placed in proper position while closed. The Tearscope Plus and a slit lamp were used at the same time to focus on the tear film of the eye and then the time from upstroke of the last blink to the first tear film break (or dry spot formation) was recorded as NIBUT (in seconds).
All animals were sacrificed (i.e., euthanized) on Day 48. No gross necropsy was performed. both eyes, superior and inferior eyelids of animals taken out and fixed 10% neutral buffered formalin.
   The results of the NIBUT measurements obtained from meibomian gland dysfunction rabbit model are summarized in FIG. 1. FIG. 1 shows significant improvement in NIBUT of cauterized rabbit eyes after administration of the emulsion composition of Table 1.

The graph in FIG. 1 illustrates a quantifiable benefit in using the hyaluronic acid emulsion (with lipids/oil) composition (Table 1) for improving tear film stability in the model. Specifically, FIG. 1 shows a prevention of decreasing (or worsening or decreasing trend) of tear film break-up time (as observed by the leveling off of the decrease in tear break-up due to Rabbits' dysfunctional meibomian gland in FIG. 1) and also shows a directional increase in tear film break-up time after, in each case, after the commencement of the 3 times per day administration of 30 μL of the composition of Table 1 containing 0.3% of an oil phase (i.e., castor oil). The directional increase led to an increase in tear film break-up time of 1.3 second increase (or 18% increase) at Day 40 and 1.6 second increase (or 22% increase) by Day 48 (25 and 33 days, respectively, after commencement of the 3 times per day administration of the composition of Table 1) versus the NIBUT as measured on day of commencement of administration (i.e., using the first NIBUT measurement of the day as the “measurement obtained the day (or about the time—e.g., ±1 hour) of commencement of the methods of the present invention” as described above).

When compared to the non-treated, meibomian gland-obstructed eye as measured at the same time point, there was about 4 seconds improvement in the average NIBUT measurement in the treated eye by Day 40 which measurement was maintained through Day 48 (i.e., a 100% increase of such average NIBUT measurements through the end of the study versus the average NIBUT measurements in the non-treated, meibomian gland-obstructed eye).

### EMBODIMENTS OF THE PRESENT INVENTION

1. A method of treating, preventing, relieving or reducing the symptoms associated with tear film deficiency and/or tear film instability in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, optionally, which treatment, prevention, relief or reduction is observed within (or after) about 1 day or more, 10 days or more, 20 days or more, or 30 days or more, after such administration.
2. A method of increasing lacrimal secretion in a subject in one or both eyes of need thereof, comprising the step of administering a composition comprising an oil-in-water emulsion to the eye(s) and/or lacrimal gland(s) of the subject wherein the oil phase is present at a concentration of from about 0.01% w/v to about 10% w/v based on the total composition, optionally, which increase can be observed within (or after) about 1 day or more, 10 days or more, 20 days or more, or 30 days or more, after such administration.
3. A method of increasing meibomian gland secretion in one or both eyes of a subject in need thereof, comprising the step of administering a composition comprising an oil-in-water emulsion to the eye(s) and/or the meibomian gland(s) of the subject wherein the oil phase is present at a concentration of from about 0.01% w/v to about 10% w/v based on the total composition, optionally, which increase can be observed within (or after) 1 day or more, about 10 days or more, 20 days or more, or 30 days or more, after such administration.
4. A method of increasing tear film break up time in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, optionally, which increase can be observed within (or after) 1 day or more, about 10 days or more, 20 days or more, or 30 days or more, after such administration.
5. A method of treating, reducing, relieving or preventing low, decreased or decreasing tear film break up times in one or both eyes of a subject in need thereof due to meibomian gland dysfunction, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, optionally, which treatment, prevention, relief or reduction can be observed within (or after) 1 day or more, about 10 days or more, 20 days or more, or 30 days or more, after such administration.
6. A method of treating, reducing, relieving or preventing evaporative dry eye due to reduced or the inhibition or blockage of meibomian gland secretions in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, optionally, which treatment, prevention, relief or reduction can be observed within (or after) about 1 day or more, 10 days or more, 20 days or more, or 30 days or more, after such administration.
7. A method of preventing, reducing, relieving and/or treating meibomian gland and/or lachrymal gland dysfunction in one or both eyes of a subject in need thereof, comprising the step of administering a composition comprising an oil-in-water emulsion to the eye(s), the meibomian gland(s) and/or lachrymal glands of the subject wherein the oil phase is present at a concentration of from about 0.01% w/v to about 10% w/v based on the total composition, optionally, which treatment, prevention or reduction can be observed within (or after) about 1 day or more, 10 days or more, 20 days or more, or 30 days or more, after such administration.
8. A method of preventing, reducing, relieving and/or treating evaporative dry eye symptoms due to meibomian gland disfunction, lacrimal gland dysfunction and/or tear film instability in one or both eyes of a subject in need thereof, comprising the step of administering a composition comprising an oil-in-water emulsion to the eye(s), the meibomian gland(s) and/or lachrymal glands of the subject wherein the oil phase is present at a concentration of from about 0.01% w/v to about 10% w/v based on the total composition, optionally, which treatment, prevention or reduction can be observed within (or after) about 10 days or more, 20 days or more, or 30 days or more, after such administration..
9. The method of any of the preceding embodiments (or combinations thereof), wherein the oil phase comprises an oil or oily component selected from Castor oil, Coconut oil, Cod-liver oil, Corn oil, Olive oil, Peanut oil, Safflower oil, Soybean oil, Jojoba oil, silicone oil, Sunflower oil and mixtures thereof.
10. The method of any of the preceding embodiments (or combinations thereof), wherein the oil or oily component is Castor oil.
11. The method of any of the preceding embodiments (or combinations thereof), further comprising a demulcent.
12. The method of any of the preceding embodiments (or combinations thereof), wherein the demulcent is selected from a hyaluronic acid compound, carboxymethylcellulose, carboxy methyl cellulose methylcellulose, hydroxyethylcellulose, hydroxypropylcellulose, methyl ethyl cellulose, dextran 70, gelatin, polyethylene glycols, polyvinyl alcohol, povidone and the like and mixtures thereof
13. The method of any of the preceding embodiments (or combinations thereof), wherein the demulcent is a hyaluronic acid compound.
14. The method of any of the preceding embodiments (or combinations thereof), wherein the hyaluronic acid compound is hyaluronic acid, any of its hyaluronate salts or mixtures thereof.
15. The method of any of the preceding embodiments (or combinations thereof), wherein the hyaluronic acid compound is hyaluronic acid.
16. The method of any of the preceding embodiments (or combinations thereof), wherein the demulcent is present in an amount of from about 0.01% w/v to about 5% w/v of the total composition.
17. The method of any of the preceding embodiments (or combinations thereof), wherein the composition further comprises surfactant.
18. The method of any of the preceding embodiments (or combinations thereof), wherein the surfactant is present in an amount from at least about 0.01 w/w % to no more than 5 w/w %.
19. The method of any of the preceding embodiments (or combinations thereof), wherein the surfactant is selected from one or more of tocopherol polyethyleneglycol-succinate, PEG hydrogenated Castor oil, trihydroxystearate ester of polyethoxylated glycerol, hydroxystearate (bis)ester of mixed polyethylene glycols and mixtures thereof.
20. The method of any of the preceding embodiments (or combinations thereof), wherein the surfactant is selected from two or more of tocopherol polyethyleneglycol-succinate, PEG hydrogenated Castor oil, trihydroxystearate ester of polyethoxylated glycerol, hydroxystearate (bis)ester of mixed polyethylene glycols and mixtures thereof.
21. The method of any of the preceding embodiments (or combinations thereof), wherein the surfactant is PEG hydrogenated Castor oil.
22. The method of any of the preceding embodiments (or combinations thereof), wherein the PEG hydrogenated castor oil to Castor oil is in the range of 0.6 to 0.8 PEG hydrogenated castor oil for 1.0 w/w % Castor oil, or about 0.8 PEG hydrogenated castor oil for 1.0 w/w % Castor oil.
23. The method of any of the preceding embodiments (or combinations thereof), wherein the composition comprises 0.3% of an oil phase, which may comprise, consist or consist essentially of be castor oil.
24. The method of any of the preceding embodiments (or combinations thereof), wherein the step of administering comprises the step of administering an eye drop to the eyes in need thereof.
25. The method of embodiment 24, wherein the eye drop is administered at least once per day, at least twice per day, at least 3 times per day, 3 times per day, at least four times per day, at least five per day, at least six times per day, or as needed to the eyes in need thereof
26. The method of embodiment 24, wherein the eye drop is administered every 12 hours, every 8 hours, every 6 hours every 4 hours, or as needed.
27. The method of embodiment 24, wherein the eye drops may be selected from multipurpose cleaning, rinsing, disinfecting and storage compositions, rewetting compositions, in-the-eye cleaning compositions.
28. The method of any of the preceding embodiments (or combinations thereof) wherein the step of administering comprises administering a contact lens that has been soaked in the composition prior to administration.
29. The method of embodiment 28, wherein the contact lens is a hydrogel contact lens or a silicone hydrogel contact lens.
30. The method of embodiments 28 or 29, where the contact lens is a daily disposable contact lens.
31. The method of embodiment s 28 or 29, where the contact lens is a reusable contact lens and the contact lens is soaked in a composition used in any of the foregoing claims.
32. The method of any of the preceding embodiments (or combinations thereof) wherein the treatment, prevention, relief or reduction produces an increase in non-invasive tear film break-up time of at least about 10%, about 15% or about 20% compared to the eye without any treatment with the compositions of the present invention.
33. The method of any of the preceding embodiments (or combinations thereof) wherein the treatment, prevention, relief or reduction produces an increase in NITBUT of up to about 1 seconds, at least about 1 second, about 2 seconds, at least about 2 seconds compared to the eye without any treatment with the compositions.
34. The method of any of the preceding embodiments (or combinations thereof) wherein the treatment, prevention, relief or reduction produces an increase in NITBUT of at least 10% or more, at least 15% or more, at least 20% compared to an untreated eye after one day, about 10 days, about 20 days, or about 30 days of administration of the composition.
35. The method of any of the preceding embodiments (or combinations thereof) wherein the treatment, prevention, relief or reduction produces an increase in NITBUT which treatment, prevention or reduction is observed within (or after) about 2 days, 3 days, 4 days, 5 days, 6 day, 7 days, 8 days, 9 days, 15 days, 25 days, of starting administration of the composition.
36. The method of any of the preceding embodiments (or combinations thereof) wherein the treatment, prevention, relief or reduction prevents, treats or reduces tear film deficiencies and contact lens water loss by providing an oil layer at the air-tear interface or additionally at the contact lens-tear interface when a contact lens is present.

1. A method of treating, preventing, relieving or reducing the symptoms associated with tear film deficiency and/or tear film instability in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, which treatment, prevention, relief or reduction is observed within about 1 day or more, 10 days or more, 20 days or more, or 30 days or more, after such administration.

2. A method of increasing lacrimal secretion in a subject in one or both eyes of need thereof, comprising the step of administering a composition comprising an oil-in-water emulsion to the eye(s) and/or lacrimal gland(s) of the subject wherein the oil phase is present at a concentration of from about 0.01% w/v to about 10% w/v based on the total composition, which increase can be observed within about 1 day or more, 10 days or more, 20 days or more, or 30 days or more, after such administration.

3. A method of increasing meibomian gland secretion in one or both eyes of a subject in need thereof, comprising the step of administering a composition comprising an oil-in-water emulsion to the eye(s) and/or the meibomian gland(s) of the subject wherein the oil phase is present at a concentration of from about 0.01% w/v to about 10% w/v based on the total composition, which increase can be observed within 1 day or more, about 10 days or more, 20 days or more, or 30 days or more, after such administration.

4. A method of increasing tear film break up time in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, which increase can be observed within 1 day or more, about 10 days or more, 20 days or more, or 30 days or more, after such administration.

5. A method of treating, reducing, relieving or preventing low, decreased or decreasing tear film break up times in one or both eyes of a subject in need thereof due to meibomian gland dysfunction, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, which treatment, prevention, relief or reduction can be observed within 1 day or more, about 10 days or more, 20 days or more, or 30 days or more, after such administration.

6. A method of treating, reducing, relieving or preventing evaporative dry eye due to reduced or the inhibition or blockage of meibomian gland secretions in one or both eyes of a subject in need thereof, comprising the step of administering a composition comprising an oil-in-water emulsion to the eye(s), lacrimal gland(s) and/or the meibomian gland(s) of the subject wherein the oil phase is present at a concentration of from about 0.01% w/v to about 10% w/v based on the total composition, which treatment, prevention, relief or reduction can be observed within about 1 day or more, 10 days or more, 20 days or more, or 30 days or more, after such administration.

7. A method of preventing, reducing, relieving and/or treating meibomian gland and/or lacrimal gland dysfunction in one or both eyes of a subject in need thereof, comprising the step of administering a composition comprising an oil-in-water emulsion to the eye(s), the meibomian gland(s) and/or lacrimal glands of the subject wherein the oil phase is present at a concentration of from about 0.01% w/v to about 10% w/v based on the total composition, which treatment, prevention or reduction can be observed within about 1 day or more, 10 days or more, 20 days or more, or 30 days or more, after such administration.

8. A method of preventing, reducing, relieving and/or treating evaporative dry eye symptoms due to meibomian gland disfunction, lacrimal gland dysfunction and/or tear film instability in one or both eyes of a subject in need thereof, comprising the step of administering a composition comprising an oil-in-water emulsion to the eye(s), the meibomian gland(s) and/or lacrimal glands of the subject wherein the oil phase is present at a concentration of from about 0.01% w/v to about 10% w/v based on the total composition, which treatment, prevention or reduction can be observed within about 10 days or more, 20 days or more, or 30 days or more, after such administration.

9. The method of claim 1, wherein the oil phase comprises an oil or oily component selected from Castor oil, Coconut oil, Cod-liver oil, Corn oil, Olive oil, Peanut oil, Safflower oil, Soybean oil, Jojoba oil, silicone oil, Sunflower oil and mixtures thereof.

10. The method of claim 9, wherein the oil or oily component is Castor oil.

11. The method of claim 1, further comprising a demulcent.

12. The method of claim 11, wherein the demulcent is selected from a hyaluronic acid compound, carboxymethylcellulose, carboxy methyl cellulose methylcellulose, hydroxyethylcellulose, hydroxypropylcellulose, methyl ethyl cellulose, dextran 70, gelatin, polyethylene glycols, polyvinyl alcohol, povidone and the like and mixtures thereof.

13. The method of claim 12, wherein the demulcent is a hyaluronic acid compound.

14. The method of claim 13, wherein the hyaluronic acid compound is hyaluronic acid, any of its hyaluronate salts or mixtures thereof.

15. The method of claim 14, wherein the hyaluronic acid compound is hyaluronic acid.

16. The method of claim 11, wherein the demulcent is present in an amount of from about 0.01% w/v to about 5% w/v of the total composition.

17. The method of claim 1, wherein the composition further comprises surfactant.

18. The method of claim 17, wherein the surfactant is present in an amount from at least about 0.01 w/w % to no more than 5 w/w %, based on the total composition.

19. The method of claim 18, wherein the surfactant is selected from one or more of tocopherol polyethyleneglycol-succinate, PEG hydrogenated Castor oil, trihydroxystearate ester of polyethoxylated glycerol, hydroxystearate (bis)ester of mixed polyethylene glycols and mixtures thereof.

20. The method of claim 19, wherein the surfactant is selected from two or more of tocopherol polyethyleneglycol-succinate, PEG hydrogenated Castor oil, trihydroxystearate ester of polyethoxylated glycerol, hydroxystearate (bis)ester of mixed polyethylene glycols and mixtures thereof.

21. The method of claim 20, wherein the surfactant is PEG hydrogenated Castor oil.

22. The method of claim 21, wherein the PEG hydrogenated castor oil to Castor oil is in the range of 0.6 to 0.8 PEG hydrogenated castor oil for 1.0 w/w % Castor oil, or about 0.8 PEG hydrogenated castor oil for 1.0 w/w % Castor oil.

23. The method of claim 1, wherein the composition comprises 0.3% of an oil phase, which may comprise, consist or consist essentially of be castor oil.

24. The method of claim 1, wherein the step of administering comprises the step of administering an eye drop to the eyes in need thereof.

25. The method of claim 24, wherein the eye drop is administered at least once per day, at least twice per day, at least 3 times per day, 3 times per day, at least four times per day, at least five per day, at least six times per day, or as needed to the eyes in need thereof.

26. The method of claim 24, wherein the eye drop is administered every 12 hours, every 8 hours, every 6 hours every 4 hours, or as needed.

27. The method of claim 24, wherein the eye drops may be selected from multipurpose cleaning, rinsing, disinfecting and storage compositions, rewetting compositions, in-the-eye cleaning compositions.

28. The method of claim 1, wherein the step of administering comprises administering a contact lens that has been soaked in the composition prior to administration.

29. The method of claim 28, wherein the contact lens is a hydrogel contact lens or a silicone hydrogel contact lens.

30. The method of claim 28 or 29, where the contact lens is a daily disposable contact lens.

31. The method of claim 28 or 29, where the contact lens is a reusable contact lens and the contact lens is soaked in a composition used in any of the foregoing claims.

32. The method of claim 1, wherein the treatment, prevention, relief or reduction produces an increase in non-invasive tear film break-up time of at least about 10%, about 15% or about 20% compared to the eye without any treatment with the compositions of the present invention.

33. The method of claim 1, wherein the treatment, prevention, relief or reduction produces an increase in NITBUT of up to about 1 seconds, at least about 1 second, about 2 seconds, at least about 2 seconds compared to the eye without any treatment with the compositions.

34. The method of claim 1, wherein the treatment, prevention, relief or reduction produces an increase in NI I BUT of at least 10% or more, at least 15% or more, at least 20% compared to an untreated eye after one day, about 10 days, about 20 days, or about 30 days of administration of the composition.

35. The method of claim 1, wherein the treatment, prevention, relief or reduction produces an increase in NITBUT which treatment, prevention or reduction is observed within about 2 days, 3 days, 4 days, 5 days, 6 days, 7 days, 8 days, 9 days, 15 days, or 25 days of starting administration of the composition.

36. The method of claim 1, wherein the treatment, prevention, relief or reduction prevents, treats or reduces tear film deficiencies and contact lens water loss by providing an oil layer at the air-tear interface or additionally at the contact lens-tear interface when a contact lens is present.
